(19) United States
(12) Patent Application Publication
(10) Pub. No.: US 2011/0176960 A1
(43) Pub. Date: Jul. 21, 2011

(54) Decontaminating catalyst-reagent system and method

(76) Inventor: Michael L. Rowzee, West Jordan, UT (US)

(21) Appl. No.: 12/657,216

(22) Filed: Jan. 15, 2010

(51) Int. Cl. A61L 2/16

(52) U.S. Cl. 422/37

(57) ABSTRACT

A decontaminating catalyst-reagent system and method for abating chemical and microbial hazards on living mammals and non-living materials.

### CROSS-REFERENCE TO RELATED APPLICATIONS

U.S. Pat No. RE37,207; Cronce; Jun. 5, 2001
U.S. Pat No. 6,376,436; Cronce; Apr. 23, 2002
U.S. Pat No. 6,566,574; Tadros, et al; May 20, 2003
U.S. Pat No. 6,623,695; Malchesky, et al; Sep. 23, 2003
U.S. Pat No. 6,797,681; Fricker, et al; Sep. 28, 2004
U.S. Pat No. 7,371,714; Johnston, et al; May 13, 2008
U.S. Pat No. 7,531,132; Delcomyn, et al; May 12, 2009
Primary Examiner: Michael L. Rowzee; Assistant Examiner: N/A; Attorney/Agent: N/A

### OTHER REFERENCE MATERIAL

Hazardous Chemical Spill Cleanup, Noyes Data Corp., 1979
U.S. Army Field Manual 3-5, NBC Decontamination, 28 Jul. 2000
U.S. Army Field Manual 3-11, Multiservice Tactics, Techniques, and Procedures for Nuclear,
   Biological, and Chemical Defense Operations, Jan. 12, 2006
Nucleophilic Substitution at a Saturated Carbon Atom, Elsevier Publishing Company, 1963
Organic Reaction Mechanisms, W. A. Benjamin Publishers, 1969
Advanced Organic Chemistry, Reactions and Mechanisms, Prentice Hall, 1998
Surfactants and Interfacial Phenomena, Wiley & Sons, Inc. 1989
Catalysis in Micellar and Macromolecular Systems, Academic Press, 1975
Handbook of Reactive Chemical Hazards, Butterworths, 1981
Hazardous Materials Chemistry for Emergency Responders, Lewis Publishers, 1997

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Definitions:

The terminology “comprised of” has the same meaning as “comprising” and is intended herein to leave the claim open to additional elements. The terminology “may” has the same meaning as “may also,” “could” and “can” and is not intended herein as a claim-closed terminology. The terminologies “include,” “including” and “includes” have the same meaning as “including but not limited to” and is not intended herein as a claim-closed terminology.

### Chemical Reactions

Chemical reactions in laboratories normally occur with known chemicals and conditions. The decontamination of hazardous materials often takes place in uncontrolled environments where many residues are unknown and conditions are uncontrollable. The purpose of decontamination by chemical reaction is to change the properties of contaminants from hazardous to less-than-hazardous. In field decontamination, thoroughly understanding the types of chemical reactions taking place is usually unnecessary. The goal of decontamination is not understanding, it is detoxification.

### Mammals

Humans, pets, livestock and other animals may be exposed to toxic chemical residues and require decontamination. The present protocol in the existing art calls for the use of 0.5% sodium hypochlorite to decontaminate living tissue. Higher concentrations are often needed but the likelihood of tissue damage dictates using a lower concentration on living tissue. Where decontamination does not occur quickly enough, the exposure may result in permanent damage or death. What is lacking in the existing art is a decontaminating formulation that will detoxify tissue as quickly as high-concentrations of corrosive formulations but will not destroy living tissue.

### Dwellings

The interior of a dwelling is a difficult environment to decontaminate. A variety of residues, polar and non-polar, are found on indoor surfaces. Soot and residue of polycyclic aromatic hydrocarbons accumulate on surfaces from indoor sources such as cooking, heating and smoking, and from outdoor sources such as auto exhaust and industrial emissions. A large percentage of the dust in a home is human skin. Human skin is an oily material and a host to bacteria, fungi, dust mites, etc.

When people cook oily foods or fry foods in oil, aerosolized oil accumulates on indoor surfaces, especially in cooking areas. In buildings, unwanted residue may be found on paint, metal, wood, plastic, fabric, sheetrock, and other surfaces. Surfaces may be soiled, clean, oily, absorbent, nonabsorbent, smooth, rough, cold, hot, etc. Residue may be found on the top of surfaces, beneath paint or oil, wet, dry, diluted, concentrated, or blended with other chemicals.

Certified Decontamination, a Utah company (my company), used a decontaminating protocol for many years that badly corroded metal, damaged electrical parts, warped floors, ruined cabinets, and significantly damaged plaster, sheetrock, and paint. Where residue levels were below toxic levels but above statutory limits, one could say, “The cure was almost worse than the disease.” The regrettable damage was the motivation to research and develop this invention.

### Clandestine Drug Manufacturing

In the United States, jurisdictions such as states and health departments have, on the most part, adopted a strategy of decontamination by washing, demolishing, or encapsulating for situations such as clandestine meth labs and other drug-contaminated properties. Efforts to decontaminate by washing are often unsuccessful. A second, very common method is decontamination by partial demolition. The decontamination contractors remove and discard all furniture, property, flooring, furnaces, ducts, walls, ceilings, cabinets, and appliances.

Washing and decontamination strategies in meth-contaminated dwellings often result in partial decontaminations. An example of a partial decontamination is where a clandestine meth lab is operated in an unfinished basement. Some contractors would remove sheetrock, flooring, cabinets, furnace, ducts, etc., then paint over the basement wall, floor, joists, and other remaining materials. In other words, they remove the least-contaminated materials, keep but paint over the most-contaminated materials, and claim the dwelling is safe for habitation. A few jurisdictions allow decontamination by only painting. Typical encapsulation is the painting of indoor materials with a sealer-type paint. A family can add two gallons of moisture to the indoor air everyday. Even in dry climates, the humidity can be sufficient to leach toxic chemicals through the paint where they off-gas into the indoor air.

Many home builders and home owners seal doors and windows to prevent cold air from entering during winter months. When a gas furnace ignites, indoor air is consumed and escapes the house up the flue. As a result, the dwelling interior becomes a vacuum. Replacement air enters through any available opening even cracks in walls, floors, etc. Gases from encapsulated hazardous materials can be pulled into the indoor environment. Even environments with low humidity and where no burning appliances are indoors, volatile forms of mercury and iodine cannot be contained with paint, even sealer and epoxy paint.

### BRIEF SUMMARY OF THE INVENTION

This invention relates to the decontamination of chemical and biological residues in military, civil, commercial, and residential environments. In addition to decontaminating chemical and biological warfare (CBW) agents and easily-decontaminated non-military contaminants, this invention decontaminates hydrocarbon-derivative compounds that are often difficult to decompose. In the preferred embodiment, the catalyst-reagent is combined with one or more decontaminating agents to create a decontaminating formulation. The corrosion-inhibiting component may be added to the decontaminating formulation prior to application or applied to the surface soon after decontamination.

One of the limitations of corrosive decontaminating formulations and protocols is the damage caused to living tissues, metals, organic components, electrical parts, and other materials. The dilemma is: few hazardous materials are promptly decontaminated by non-corrosive formulations but corrosive formulations can destroy the objects one is trying to decontaminate. This invention uses potentially corrosive and damaging compounds, but can accomplish decontamination goals faster or with lower concentrations. It also provides for concurrently or subsequently deactivating some or all of the corrosive nature of decontaminating agents thereby precluding or minimizing damage.

### DETAILED DESCRIPTION OF THE INVENTION

In the field environment, particularly inside dwellings, surfaces can be comprised of polar and non-polar materials. These materials can be “glued” together by oily or waxy deposits. This invention provides induced interfacial changes to breach interfacial repulsions between polar and non-polar matter. The conditions, concentrations and characteristics of the decontaminating formulation and substrate will determine the rate of interfacial change.

When the formulation of this invention is applied to a target surface, decontamination can proceed through the following stages:
1. Reduction of interfacial free energy;
2. Displacement of trapped air;
3. Adsorption (physical and chemical);
4. Rearrangement of atoms to less stressful configurations; and
5. Creation of final products.

Where multiple layers of semi-solids are adhered to the contaminated surface, repeated applications may be needed. Gel-producing and foam- producing components are not added to a formulation of this invention. Adding ingredients that cause gelling or foaming, or agitating to produce foam prior to applying the decontaminating formulation can reduce the desired interfacial phenomena, diminish displacement of trapped air, and reduce or prevent adsorption.

In the preferred embodiment, surfactant concentration in the decontaminating formulation begins below the critical micelle concentration (CMC). The CMC is a concentration range where micelles are formed around nonpolar materials. The phenomena does not occur where the surfactant concentration is above or below CMC. Decontaminating reactions can accelerate when water in the formulation evaporates and the surfactant reaches critical micelle concentration. Within the micelle pseudo-phase of the surfactant aggregates, a more favorable orientation and solvation of the substrate materials (solvent-separation) increases contact (collisions) which in turn increases stress on the affected species. The ability of the decontaminating formulation in the preferred embodiment to catalyze with micelles adds another reaction mechanism to the poly-reactive nature of this invention.

In the preferred embodiment, where feasible, the environment is prepared prior to application of the decontaminating formulation, then managed and altered until decontamination is complete. Two important preparations are cleaning and warming. Generally, clean surfaces decontaminate faster than dirty surfaces and warm residues react faster than cold residues. Where endothermic reactions will occur, heating can be essential. Managing and altering the environment during decontamination may be necessary to quickly bring reactions to completion. The environment may be managed and altered with the following measures:
1. Changing the physical state of the residue. For example, from a solid to a liquid or from a liquid to a gas. This includes suspending a solid in a liquid solution.
2. Increasing or decreasing the temperature of the surface, area, and/or decontaminating solution to bring contaminants and decontaminants to the same temperature.
3. Re-wetting contaminants and surfaces and ensuring sufficient reactants are present by applying additional decontaminating formulation.
4. Increasing the temperature of the surface to provide endothermic energy.
5. Removing one or more of the intermediate and byproduct elements and compounds produced during the chemical reaction, then reinitiating the reaction.
6. Increasing or decreasing relative humidity to speed up or slow down evaporation.

### DIFFERENCES FROM THE EXISTING ART

In the preferred embodiment, this invention differs from the existing art in ways such as the following:
1. This invention provides a catalyst-reagent to mix with one or more decontaminating agents and reduces the amount of decontaminating agents that are used.
2. This invention includes managing and altering the decontaminating environment as part of the method.
3. This invention can decontaminate human and animal skin and hair more quickly than decontaminating agents alone, and without as much damage.
4. This invention can be used to decontaminate metal components in equipment such as electronics, weapons, and vehicles without rendering the equipment inoperative.
5. Formulations of this invention are never applied as a foam or gel.
6. Rinsing is often not necessary.

### OBJECTIVES AND BENEFITS

In the preferred embodiment, the objectives and advantages of this invention include the following:
1. It will decontaminate a wide variety of contaminants including drug residues and other psychoactive compounds such as methamphetamine and 3-quinuclidinyl benzilate (BZ), pathogenic or nuisance microorganisms, and microorganism-produced chemical compounds such as mycotoxins.
2. It requires a lower concentration of corrosive chemicals and may be used to decontaminate skin, hair, non-colorfast materials, metals, electronics, and other materials that would be damaged by high concentrations of decontaminating agents.
3. It includes components to inhibit reverse reactions and reduce the possibility of toxic byproducts.
4. It will decontaminate more than one type of hazardous chemical residue at the same time on the same surface.
5. It does not require identifying the contaminant prior to use.
6. It is not flammable.
8. Impervious, protective suits are not always needed where decontamination personnel use this invention in moderately-hazardous environments.
10. It may be applied to surfaces as a protective barrier before the surfaces are contaminated.
11. Rinsing is often not necessary.

### Decontaminating Chemicals and Organisms

The decontaminating catalyst-reagent system and method is suitable to decontaminate chemical residues and pathogenic organisms. Most contaminants can be decontaminated using the preferred embodiment. Depending on the contaminant and other factors, using a decontaminating agent other than the decontaminating agent of the preferred embodiment may be desired or necessary. This invention, in the preferred embodiment or by changing the decontaminating agent, decontaminates chemical residues and pathogenic organisms including:

a. Military chemical agents such as choking agents (CG, PS); nerve agents (GA, GB, GD, GE, GF, VE, VG, VS, VX); blood agents (AC, CK, SA); blister agents (CX, ED, H, HD, HL, HT, HN-1, HN-2, HN-3, L, MD, PD, Q); incapacitating agents (BZ); respiratory irritants (DA, DC, DM, Cl2); riot control agents (CN, CR, CS, CS1, CS2, CSX, OC); chemical agent weapon precursors (DF, QL, OPA, NE, NM).

b. Pathogenic microorganisms (bacteria, fungi, viruses, etc.).

c. Microorganism-produced toxins and mycotoxins.

d. Hydrocarbon derivative compounds including:
1. Cholinergics (acetylcholine agonists) such as nicotine, and piracetam.
2. Anticholinergics (acetylcholine antagonists) such as scopolamine, hyoscyamine, dimenhydrinate, diphenhydramine, atropine, benactyzine, dicyclomine, N-ethyl-3-piperidyl benzilate, N-methyl-3-piperidyl benzilate, 3-quinuclidinyl benzilate, ditran, EA-3167, and most tricyclics
3. Dopamine reuptake inhibitors (DRIs) such as cocaine, methylphenidate, amphetamine, methamphetamine, and bupropion.
4. Dopamine agonists such as pramipexole, and 3,4-dihydroxy-L-phenylalanine (L-DOPA).
5. Dopamine receptor antagonists such as haloperidol, droperidol, and many antipsychotics.
6. GABA reuptake inhibitors such as tiagabine.
7. GABA receptor agonists such as diazepam and other benzodiazepines, ethanol, barbiturates, muscimol, and ibotenic acid.
8. GABA antagonists such as thujone, and bicuculline.
9. Norepinephrine reuptake inhibitors including most non-SSRI antidepressants such as amoxapine, atomoxetine, bupropion, venlafaxine and the tricyclics.
10. Norepinephrine releasers such as mianserin.
11. Serotonin receptor agonists such as lysergic acid diethylamide (LSD), psilocybin, mescaline, and dimethyltryptamine (DMT).
12. Serotonin reuptake inhibitors including most antidepressants including tricyclics such as amitryptyline and SSRIs such as fluoxetine and sertraline.
13. Serotonin releasers such as 3,4-methylenedioxy-N-methylamphetamine (MDMA) (ecstasy).
14. AMPA receptor antagonists such as kynurenic acid, and 2,3-dihydroxy-6-nitro-7-sulfamoyl-benzo[f]quinoxaline-2,3-dione (NBQX).
15. Cannabinoid receptor agonists such as tetrahydrocannabinol (THC).
16. Melanocortin receptor agonists such as bremelanotide.
17. NMDA receptor antagonists such as ketamine, Phencyclidine (PCP), and dextromethorphan (DXM).
18. μ-opioid receptor agonists such as morphine, heroin, and oxycodone.
19. μ-opioid receptor inverse agonists such as naloxone, and naltrexone.
20. κ-opioid receptor agonists such as salvinorin A, butorphanol, and nalbuphine.
21. κ-opioid receptor inverse agonists such as buprenorphine.
22. Monoamine oxidase inhibitors (MAOIs) such as phenelzine, and iproniazid.
23. Dermal irritants such as Urushiol (urishiol oil), anacardic acid, and pyrocatechol.
24. Polycyclic aromatic hydrocarbons (PAHs) such as benzo[a]pyrene, benzo[b]fluoranthene, benzo[ghi]perylene, benzo[j ]fluoranthene, benzo[k]fluoranthene, benz[a]anthracene, chrysene, coronene, dibenz[a,h]anthracene, indeno[1,2,3-cd]pyrene, and ovalene.
25. Putrefaction compounds such as putrescine and cadaverine.

e. Clandestine drug lab chemicals such as volatile forms of iodine, volatile forms of chlorine, red phosphorus, mercury, methamphetamine, and other residues.

I claim:

1. A decontaminating catalyst-reagent system and method for decontaminating mammals, buildings, vehicles, equipment, and materials that are contaminated with chemical residues and pathogenic organisms comprising:
a. An amphipathic component of the catalyst-reagent system and method comprising:
1. Two nonionic octylphenol ethoxylate compounds with a mole number of 6 and a mole number of 10 respectively, but may be comprised of one or more nonionic octylphenol ethoxylate compounds with mole numbers other than 6 and 10; one or more alkyl sulphate compounds, alkyl phenol ethoxylate compounds, nonylphenol ethoxylate compounds, amphoteric/zwitterionic compounds, esterquat compounds, mono alkyl quaternary compounds, quaternary ammonia compounds, crown ethers, salts of onium compounds, Rainey nickel compounds, Rainey nickel-like compounds, imidizole compounds, sodium silicate compounds, zeolite compounds, and/or other amphipathic and/or amphoteric compounds having, buffering, doping, detergent, emulsifying, solubilizing, deflocculating, phase transfer catalyzing, interfacial free energy reducing, and/or dispersing actions or propensities.
2. One or more wetting compound, surfactant compound, emulsifying compound, deflocculating compound, solubilizing compound, interfacial free energy reducing compound, and/or phase transfer catalyst.
3. A concentration of amphipathic component in an aqueous decontaminating formulation of the catalyst-reagent system and method of 0.25% by volume but may be comprised of 0.1% to 10% by volume or more if needed.
b. A reagent component of the catalyst-reagent system and method comprising:
1. Sodium tetraborate decahydrate, but may be comprised of one or more element or compound of boron, sodium borate, disodium octaborate tetrahydrate, sodium tetraborate pentahydrate, fluorine, fluorine compound, bromine, bromine compound, iodine, iodine compound, iron, soluble iron powder, iron oxide, other iron compound, and other reagent elements and compounds.
2. A concentration of reagent component in an aqueous decontaminating formulation of the catalyst-reagent system and method of 1.25% by volume but may be comprised of 0.1% to 20% by volume or more if needed.
c. A decontaminating agent component of the catalyst-reagent system and method comprising:
1. Sodium hypochlorite, but may be comprised of one or more compound of chlorine dioxide, potassium hypochlorite, calcium hypochlorite, sodium dichloro-s-triazinetrione, trichloro-s-triazinetrione, other hypochlorite or chlorate compounds, hydrogen peroxide, sodium peroxide, other peroxide compounds, sodium perborate (and its monohydrate), other sodium borate compounds, sodium percarbonate, other carbonate or percarbonate compounds, sodium hydroxide, potassium hydroxide, other hydroxide compounds, supertropical bleach, a pesticide registered with the EPA for use against the type of organism or for use in the specific type of location, other reduction/oxidation compounds, and other electrophilic and neucleophilic compounds.
2. A concentration of decontaminating agent in an aqueous decontaminating formulation of the catalyst-reagent system and method of 0.4% by volume but may be comprised of 0.001% to 99% by volume or more if needed.
d. A corrosion inhibitor component of the catalyst-reagent system and method comprising:
1. Disodium metasilicate pentahydrate for metals and non-living materials or comprising sodium thiosulfate for skin, and other live tissues; but may be comprised of one or more elements or compounds of sodium sulfite, sodium pyrophosphate, sodium polyphosphate, sodium borate, ethylene diamine tetra-acetic acid, mercapto-benzimidazole, thiabendazole, other imidazole compounds, ascorbic acid, sodium ascorbate, calcium ascorbate, potassium ascorbate, sodium ferrocyanide, soluble ferric pyrophosphate, ferric citrate, ferrous sulfate, ferric ammonium citrate, insoluble ferric orthophosphate, soluble ferric orthophosphate, ferric ammonium oxalate, ferric ammonium sulfate, ferric bromide, ferric sodium oxalate, ferric stearate, ferric sulfate, ferrous acetate, ferrous ammonium sulfate, ferrous bromide, ferrous gluconate, ferrous iodide, ferric acetate, ferric fluoroborate, ferric hydroxide, ferric oleate, ferrous fumarate, ferrous oxalate, ferrous oxide, ferric lactate, ferric resinate, sodium oxalate, oxalic acid, and other corrosion inhibiting elements or compounds.
2. A concentration of corrosion inhibitor component in an aqueous mixture of the catalyst-reagent system and method or other aqueous mixture of 0.25% but may be comprised of 0.1% to 25% by volume or more if needed.
e. Combining the catalyst-reagent with one or more decontaminating agents and with one or more corrosion inhibitors in an aqueous decontaminating formulation mixture; then applying the decontaminating formulation mixture to contaminated material while managing and altering the environment.
f. Combining the catalyst-reagent with one or more decontaminating agents to make an aqueous decontaminating formulation; then applying the decontaminating formulation to contaminated material while managing and altering the environment; then subsequently applying an aqueous mixture of one or more corrosion inhibitors to the decontaminated material.
e. Managing and altering the environment before and during decontamination comprising:
1. Changing the physical state of the residue. For example, from a solid to a liquid or from a liquid to a gas. This includes suspending a solid in a liquid solution.
2. Increasing or decreasing the temperature of the surface to bring contaminants and decontaminants to the same temperature.
3. Re-wetting contaminants and surfaces and ensuring sufficient reactants are present by applying additional decontaminating formulation.
4. Increasing the temperature of the surface to provide endothermic energy.
5. Removing one or more of the intermediate and byproduct elements and compounds produced during the chemical reaction, then reinitiating the reaction.
6. Increasing or decreasing relative humidity to speed up or slow down evaporation.
f. Applying the decontaminating formulation and corrosion inhibitor by high-pressure sprayer, but may be comprised of applying the decontaminating formulation and corrosion inhibitor by low-pressure sprayer, high-pressure/low-volume sprayer, high-volume sprayer, low-volume sprayer, fogger, wiping, blotting, brushing, mopping, splashing, immersing, dipping, or showering.
